(19) United States
(12) Patent Application Publication
(10) Pub. No.: US 2017/0016925 A1
(43) Pub. Date: Jan. 19, 2017

(54) PHARMACEUTICAL FORM FOR COMBATING CHEMICAL SUBMISSION OF A MEDICAMENT

(71) Applicant: ETHYPHARM, ST CLOUD CEDEX (FR)

(72) Inventors: CATHERINE HERRY, SAINT-OUEN DU TILLEUL (FR); PAULINE CONTAMIN, LA FEUILLIE (FR); EMMANUEL DUPAU, ROUEN (FR)

(73) Assignee: ETHYPHARM, ST CLOUD CEDEX (FR)

(21) Appl. No.: 15/281,595

(22) Filed: Sep. 30, 2016

Related U.S. Application Data
(62) Division of application No. 13/808,810, filed on Jan. 7, 2013.

(30) Foreign Application Priority Data
Jul. 6, 2010 (FR) 1055491

(51) Int. Cl. G01N 33/94; A61K 9/20; G01N 21/94; A61K 31/485; A61K 9/48; G01N 33/14; A61K 9/00; A61K 31/437

(52) U.S. Cl. CPC G01N 33/94; A61K 9/0056; A61K 9/2054; A61K 9/2018; A61K 9/2027; A61K 9/2013; A61K 9/2009; A61K 31/437; A61K 9/2077; A61K 31/485; A61K 9/4808; G01N 33/14; G01N 21/94

(57) ABSTRACT

A pharmaceutical form for combating chemical submission includes an active ingredient and at least one compound which enables immediate modification of the organoleptic characteristics of a beverage into which the pharmaceutical form is introduced. The compound is selected from the group consisting of an opacifier, a fluorescent agent, floating particles, particles that are perceptible in the mouth, effervescent microgranules, and mixtures thereof.

This application is a division of U.S. application Ser. No. 13/808,810, filed 7 Jan. 2013, which is the National Phase filing of International Appln. No. PCT/FR2011/051601, filed 5 Jul. 2011, which claims priority of French Appln. No. 10/55,491, filed 6 Jul. 2010, the entireties of which applications are incorporated herein by reference for all purposes.

### FIELD OF THE INVENTION

The subject of the invention is a pharmaceutical form for combating surreptitious administration of chemicals, and a method of using it.

### BACKGROUND OF THE INVENTION

For some years, delinquents have been using the hypnotic properties of certain substances to drug someone without their knowledge. The delinquents do not hesitate to introduce surreptitiously into their victim's drink a pharmaceutical form to alter their behavior, or indeed to render them totally incapable or amnesic. Once the victim has been deprived of all awareness, the criminal can take advantage of them: theft, rape or extortion of money. Moreover, the ingestion of such a pharmaceutical form without regard to the prescribed doses can lead to serious consequences, particularly if it is absorbed with a quantity of alcohol. The pharmaceutical form can also produce harmful interactions with other medicaments which the victim could have taken beforehand.

From the state of the art, a hypnotic, Rohypnol, is known which is widely used for illicit purposes by reason of its ease of dissolution and its imperceptible nature. The formulation of this drug has been revised to result in a tablet green on the outside and blue on the inside, film-coated and thus slow to dissolve, releasing a blue color. However, the blue coloration is only visible after a quarter hour of immersion in the liquid; the victim is thus not in a position to detect the surreptitiously introduced hypnotic if they drink their drink immediately.

Also known from the state of the art is the document WO 2005/059541 which relates to a kit for detecting drugs stealthily introduced into drinks. However, this system only protects persons provided with this kit.

It is thus vital and urgent to find a method enabling immediate detection of illicit diversion of drugs in case of surreptitious chemical administration without resorting to a detection device or kit.

An essential objective of the present invention is thus to propose a pharmaceutical form comprising at least one compound enabling immediate detection of said pharmaceutical form illicitly introduced into a drink. Another objective is to be able to detect the pharmaceutical form whatever the nature and color of the drink may be.

### BRIEF DESCRIPTION OF DRAWINGS

FIG. 1 is a photograph showing a beaker containing 250 ml of water (1) and a beaker into which an orodispersible tablet of zolpidem containing 50 mg of calcium silicate (opacifying agent) has been introduced (2).

FIG. 2 is a photograph showing a glass of coca-cola and a glass of coca-cola into which an orodispersible tablet C1 of zolpidem containing floating particles has been introduced.

FIG. 3 is a photograph showing a glass of coca-cola into which an orodispersible tablet C2 of zolpidem containing other floating particles has been introduced.

### SUMMARY OF THE INVENTION

The purpose of the present invention is to offer a new pharmaceutical form for combating surreptitious administration of chemicals. This purpose is achieved by means of a pharmaceutical formulation comprising an active principle and at least one compound enabling the immediate modification of the organoleptic properties of a drink into which the pharmaceutical form is introduced, said compound being selected from the group comprising: an opacifying agent, a fluorescent agent, floating particles, particles perceptible in the mouth, effervescent microgranules, and mixtures thereof.

The invention also relates to a method for combating surreptitious administration of chemicals comprising:
dissolution in a drink of a pharmaceutical form comprising an active principle and at least one compound enabling the immediate modification of the organoleptic properties of said drink, selected from the group comprising: an opacifying agent, a fluorescent agent, floating particles, particles perceptible in the mouth, effervescent microgranules, and mixtures thereof, and
detection of the pharmaceutical form by the immediate modification of the organoleptic properties of the drink.

The invention also relates to the utilization in a pharmaceutical form of at least one compound enabling the immediate modification of the organoleptic properties of a drink, selected from the group comprising: an opacifying agent, a fluorescent agent, floating particles, particles perceptible in the mouth, effervescent microgranules, and mixtures thereof, for combating surreptitious administration of chemicals.

According to the present invention, “surreptitious administration of chemicals” is understood to mean the administration of a psychoactive substance without the victim's knowledge for criminal or malicious purposes.

### DETAILED DESCRIPTION OF THE INVENTION

The detailed description of the invention applies equally to each of the subjects of the invention.

The pharmaceutical form according to the invention comprises an active principle and at least one compound enabling the immediate detection of said pharmaceutical form illicitly introduced into a drink. According to the invention the compound is selected from:
opacifying agents, and/or
fluorescent agents, and/or
floating particles, and/or
particles perceptible in the mouth, and/or
effervescent microgranules.

In the context of the invention, the compounds can be integrated into the pharmaceutical form singly or in combination. For example, a pharmaceutical form containing floating particles could be created, or indeed a pharmaceutical form comprising a mixture of the compounds described above could be proposed.

The pharmaceutical form is preferably an oral pharmaceutical form. However, it could be another type of pharmaceutical form which the delinquent would divert from its primary destination.

In the present invention, “immediate” is understood to mean the modification of the organoleptic properties of the drink which takes place in less than one minute, preferably in less than 30 seconds, still more preferably in less than 15 seconds, from the introduction of the pharmaceutical form into the drink.

According to another aspect of the invention, the term “immediate” can also be defined as the modification of the organoleptic properties of the drink which takes place in less than one minute, preferably in less than seconds, still more preferably in less than 15 seconds from the introduction and the stirring of the pharmaceutical form into the drink. “Stirring” is understood to mean a setting of the liquid in motion, for example by means of a straw, spoon or by movement of the vessel.

Opacifying Agents

Opacifying agents are inorganic compounds which make it possible to make drinks cloudy. These may be silicates such as magnesium silicate, aluminum silicate (in particular kaolin), magnesium aluminum silicate, calcium silicate, titanium dioxide and mixtures thereof. These compounds are generally present at the minimum in a quantity of at least 15 mg, preferably from 15 to 100 mg, more preferably from 20 mg to 60 mg and still more preferably from 25 to 40 mg. Below 15 mg, the opacity could prove more difficult to detect with the naked eye.

Advantageously, the opacifying agents integrated into an oral pharmaceutical form make it possible to render drinks into which they are introduced cloudy. These agents are particularly useful for rendering cloudy transparent and clear drinks such as water, white wine, apple juice, and spirits such as vodka, white rum . . . .

The opaque appearance of the drink appears from the first seconds after the introduction and stirring of the pharmaceutical form into said drink.

Fluorescent Agents

The pharmaceutical form can also contain a fluorescent agent in a quantity of at least 0.1 mg, preferably in a quantity of at least 1 mg, more preferably between 0.2 and 5 mg, and still more preferably between 0.3 to 2 mg. This agent can be fluorescein and derivatives thereof, or indocyanine green.

This agent is visible in all types of drink in the presence of ultraviolet rays and in the dark. It makes it possible to reveal the pharmaceutical form containing it by emitting fluorescent light which is emitted from the doped drink. This agent is particularly useful for warning the victim when they are in a dark space where it is easy stealthily to introduce a foreign body into a drink.

Floating Particles and Particles Perceptible in the Mouth

According to another aspect of the invention, the pharmaceutical form can contain floating particles and/or particles perceptible in the mouth. These particles are microgranules comprising a blank support which is insoluble, or rendered insoluble in water or in an alcoholic solution by coating with an insoluble polymer or by coating with a lipid material.

Microgranules

Microgranules rendered insoluble in water or in an alcoholic solution are understood to be a blank support consisting of materials soluble in water or in an alcoholic solution covered with at least one layer of materials insoluble in water or in an alcoholic solution and the function whereof is to limit or indeed to prevent the penetration of these said media towards the core of the support.

The blank support insoluble in water or in an alcoholic solution advantageously comprises at least one excipient of hydrophobic nature selected from: cellulose, cellulose derivatives (microcrystalline cellulose), phosphate derivatives (calcium phosphates), silica and silicate derivatives (magnesium silicate, aluminum silicate and mixtures thereof) and carnauba wax.

In the context of the present invention, a blank support soluble in water or in an alcoholic solution can also be utilized. The soluble blank support can comprise at least one excipient selected from: starch, saccharose, polyols such as mannitol or lactose and mixtures thereof.

It is essential that this soluble blank support be rendered insoluble in water or alcohol by covering it with a coating layer either of:
polymeric nature comprising at least one hydrophobic polymer and possibly an inert filler and/or a plasticizer and/or a surfactant,
or lipid nature comprising at least one lipid material.

In the context of the present invention, the insoluble blank support can also be covered with at least one coating layer as described above, provided that this does not disadvantageously increase the density of the particles.

The coating ratio represents the ratio between the quantity of dry mass constituting the coating layer over the total mass of the microgranule before coating (as dry mass). The coating ratio lies between 0.1% to 50% m/m, preferably from 2% to 30% m/m, and still more preferably from 5% to 40% m/m.

The coating ratio is such that the particles obtained have a density less than that of the drink into which they are to be introduced, preferably a density less than 1, such that they remain on the surface of the drink into which they are to be introduced. Such particles are called floating particles.

Polymeric Coating Layer:

The hydrophobic polymer utilized to ensure the insoluble nature of the microparticles is selected from the following group of products: non-water-soluble cellulose derivatives, (meth)acrylic (co)-polymer derivatives, polyvinyl acetate derivatives and mixtures thereof. More preferably, the hydro-phobic polymer(s) is (are) selected from the following group of products: ethylcellulose, cellulose acetate butyrate, cellulose acetate, the type A and type B ammoniomethacrylate copolymers sold under the trade name Eudragit®, in particular Eudragit® RS 30D, Eudragit® NE 30D, Eudragit® RL 30D, Eudragit® RS PO and Eudragit® RL PO of the poly(ethyl acrylate, methyl methacrylate, trimethylammonioethyl methacrylate) family, polyvinyl acetates and mixtures thereof.

The quantity of hydrophobic polymer lies between 50% to 100%, preferably from 70% to 100%, of the dry mass of the coating layer.

An inert filler can be present in the coating layer in a proportion from 0 to 50% m/m, preferably from 0 to 20% m/m and still more preferably from 5 to 20% of the dry mass of the hydrophobic coating polymer.

The inert filler uniformly distributed in the coating is selected from the group comprising in particular talc, anhydrous colloidal silica, magnesium stearate, glycerol monostearate and mixtures thereof.

When the coating is effected by an aqueous route, a plasticizer can be added to the coating dispersion in a proportion from 0% to 50% m/m, preferably from 2% to 25% m/m, in dry mass of hydrophobic coating polymer.

The plasticizer is in particular selected from the following group of products: glycerol and esters thereof, preferably from the following subgroup: medium-chain triglycerides, acetylated glycerides, glyceryl monostearate, glyceryl triacetate, glyceryl tributyrate, phthalates, preferably from the following subgroup: dibutyl phthalate, diethyl phthalate, dimethyl phthalate and dioctyl phthalate, citrates, preferably from the following subgroup: acetyl tributyl citrate, acetyl triethyl citrate, tributyl citrate and triethyl citrate, sebacates, preferably from the following subgroup: diethyl sebacate and dibutyl sebacate, adipates, azelates, benzoates, chlorobutanol, polyethylene glycols, plant oils, fumarates, preferably diethyl fumarate, malates, preferably diethyl malate, oxalates, preferably diethyl oxalate, succinates, preferably dibutyl succinate, butyrates, esters of cetyl alcohol, malonates, preferably diethyl malonate, castor oil (the latter being particularly preferred), and mixtures thereof.

More preferably, the plasticizer is selected from the following group of products: acetylated mono-glycerides, in particular Myvacet® 9-45, triethyl citrate (TEC), dibutyl sebacate, triacetin, and mixtures thereof.

The surfactant is optionally present in the coating in a proportion of 0 to 30% m/m, preferably from 0 to 20% m/m, and, still more preferably, from 5 to 15% of the dry mass of plasticizer. The surfactant is preferably selected from the following group of products: alkali or alkaline earth metal salts of fatty acids, sodium dodecyl sulfate and sodium docusate being preferred, polyethoxylated oils, preferably polyethoxylated hydrogenated castor oil, polyoxyethylene-polyoxypropylene copolymers, poly-ethoxylated sorbitan esters, polyethoxylated castor oil derivatives, stearates, preferably of calcium, magnesium, aluminum or zinc, polysorbates, stearyl-fumarates, preferably of sodium, glycerol behenate, benzalkonium chloride, acetyltrimethylammonium bromide, cetyl alcohol and mixtures thereof.

Lipid Coating Layer:

The microgranules can also be coated by coating with a lipid material.

The lipid material according to the invention is in particular selected from the following group of products: glyceryl palmitostearate, waxes, polyoxyl-glycerides and glyceryl behenate.

The quantity of lipid material lies between 50 and 100%, preferably between 80 and 100%, of the dry mass of the coating layer.

The quantity of lipid material is selected such that the density of the resulting particles is less than that of the drink into which they are to be introduced, preferably a density less than 1, such that they remain on the surface of the drink into which they are to be introduced.

The floating particles exhibit a total diameter (blank support, optionally coated if necessary) lying between 50 and 500 μm, preferably between 200 and 500 μm so as not to be perceptible in the mouth and to ensure some comfort to the patient. On the other hand, the particles perceptible in the mouth exhibit a total diameter greater than 500 μm, preferably greater than 1 mm, so as to be perceived by the lips and above all by the taste buds. The diameter of the floating particles and those perceptible in the mouth is measured by dry method laser granulometry (Malvern laser granulometer: Mastersizer 2000).

Entirely advantageously, said particles perceptible in the mouth are floating particles.

The quantity of the particles which float and/or are perceptible in the mouth contained in the pharmaceutical form is at least 25 mg, preferably 40 mg.

Preferably, the floating particles and/or particles perceptible in the mouth can be colored by means of at least one of the following coloring agents: indigocarmine, erythrosine, brilliant blue FCF, alphazurine FG, fast green FCF, quinzarin green SS, orange II, tartrazine, sunset yellow FCF and/or can be rendered fluorescent by means of a fluorescent agent selected from the group comprising fluorescein and derivatives thereof and indocyanine green.

Advantageously, the active principle can also be colored with at least one colorant as described above so as to prevent possible sorting between the active principle and the floating particles and/or the particles perceptible in the mouth.

Also advantageously, the particles which float and/or are perceptible in the mouth are suitable for all types of drink.

From the introduction of the pharmaceutical form into the drink, the floating particles immediately rise to the surface of the drink and are visible to the naked eye. These particles remain on the surface of the liquid for at least 5 minutes and preferably for at least 4 hours, more preferably for at least 12 hours.

The particles perceptible in the mouth can also be floating particles. These are detected immediately by the victim on taking the first mouthful of the doped drink.

Effervescent Microgranules

The solid pharmaceutical form can also contain effervescent microgranules. The effervescent microgranules contain a basic excipient which will create an effervescence when it is in the presence of an acidic drink of the soda or beer type.

According to a first aspect, the microgranules comprise a blank support (soluble, insoluble or rendered insoluble) coated with particles of an alkaline agent selected from the group comprising sodium bicarbonate, calcium carbonate, and mixtures thereof.

The quantity of alkaline agent is at least greater than 5 mg, preferably greater than 10 mg and still more preferably greater than 20 mg.

When the pharmaceutical form containing the effervescent microgranules is introduced into an acidic drink, the particles of alkaline agent(s) on contact with the acid present create an effervescence visible to the naked eye.

According to a particular embodiment of the invention, the effervescent microgranules may be coated. The coating is sufficiently permeable to allow the release of particles of effervescent agent over a period of at least thirty minutes to one hour. The coating contains at least one insoluble polymer of the family of cellulose derivatives, vinyl derivatives or acrylic derivatives. It can contain a plasticizer and/or a surfactant. It can be permeabilized by addition of a soluble porogenic agent such as for example soluble derivatives of cellulose, povidone or a disintegrating agent.

The quantity of effervescent microgranules contained in the pharmaceutical form is at least 25 mg, preferably 40 mg.

Preferably, the effervescent microgranules can be colored by means of at least one coloring agent selected from indigocarmine, erythrosine, brilliant blue FCF, alphazurine FG, fast green FCF, quinzarin green SS, orange II, tartrazine, sunset yellow FCF and/or can be rendered fluorescent by means of a fluorescent agent selected from the group comprising fluorescein and derivatives thereof and indocyanine green.

Thus, a colored effervescence and/or effervescence will appear on the surface of the drink after introduction of the pharmaceutical form containing said microgranules.

Active Principle

The invention is suitable for any active principle which modifies the patient's state of consciousness. More particularly, the active principle is selected from the group comprising: anxiolytics for example the benzodiazepines, hypnotics, sedatives, and analgesics for example of the opioid type.

The anxiolytics are a class of psychotropic drugs, preferably selected from Alprazolam, Bromazepam, Chlordiazepoxide, Clobazam, Clonazepam, Clotiazepam, Clorazepate, Diazepam, Estazolam, Flunitrazepam, Lorazepam, Lormetazepam, Midazolam, Nitrazepam, Nordazepam, Oxazepam, Prazepam, Temazepam, Tetrazepam, Triazolam, clozapine, olanzapine, pirenzepine, zolpidem, lezopiclone, zaleplon, meprobamate, etifoxine and mixtures thereof.

The opioids are preferably selected from Alfentanil, Anileridine, Butorphanol, carfentanil, Codeine, Diamorphine (heroin), Dextropropoxyphene, the Encephalins, the Endorphins, Fentanyl, Hydrocodone, Hydromorphone, Methadone, Morphine, Nalbuphine, Oxycodone, Oxymorphone, Pentazocine, Pethidine (meperidine), Propoxyphene, Remifentanil, Sufentanil, Tramadol and Buprenorphine, and mixtures thereof.

According to a particular aspect of the invention, the active principle present in the pharmaceutical form is in solid form.

According to a particular embodiment, the active principle can also be colored by means of at least one coloring agent. The coloring agent can be one of those described above and/or can be rendered fluorescent by addition of a fluorescent agent such as described above.

According to another embodiment, the active principle can be coated onto the particles which float and/or are perceptible in the mouth.

According to a particular mode of the invention and apart from the compounds which are described above and which make it possible to combat surreptitious administration of chemicals, the pharmaceutical form can also contain within its matrix at least one water-soluble coloring agent selected from the group comprising: indigocarmine or E 132, erythrosine or E 127, brilliant blue FCF, alphazurine FG, fast green FCF, quinzarin green SS, orange II, tartrazine and sunset yellow FCF. The water-soluble coloring agents that can be utilized in the invention are colorants soluble in any liquid at least in part containing water and which are pharmaceutically acceptable.

The coloring agent is present in a quantity sufficient to enable a coloration intense enough to be perceived with the naked eye and which can appear from the first seconds after the introduction and stirring of the pharmaceutical form into said drink. Thus, the coloring agent is present in a proportion of at least 0.05 mg, preferably from 0.2 to 5 mg, and still more preferably from 0.3 to 2 mg in the pharmaceutical form.

When the coloring agent is indigocarmine, an intense blue color is released immediately from the pharmaceutical form, coloring for example the drink blue if it is a colorless drink such as water or lemonade or green if it is a yellow drink such as orange juice.

Erythrosine, another coloring agent, colors the drinks red.

Drink

In the present application, the term drink will be used to designate cold drinks and hot drinks, for example water, sparkling water, wine (red, white or rosé), beer (brown or light), liqueurs, spirits such as vodka, rum, brandy, tequila, whisky, cocktails, fruit juices such as orange juice or grape juice, sodas such as coca-cola or lemonade, coffee or tea. These drinks are given as an indication but in no way restrictively.

In the present invention, the vessel containing a drink into which the pharmaceutical form may be introduced has a capacity lying between 3 cl and 1 L.

The Production Process

Depending on the nature of the active principle, it can be in the form of microcrystals, microgranules or brought into suspension and coated onto a blank support.

When it is coated onto a blank support, the active principle is in the form of a solution or suspension in an aqueous or organic solvent. A binder, a diluent and/or an antistatic agent can also be added.

The blank support can be any chemically and pharmaceutically inert excipient, existing in particulate, crystalline or amorphous form. By way of example, derivatives of sugars such as lactose or saccharose, hydrolyzed starch (maltodextrins) or also celluloses, are cited. Mixtures such as saccharose and starch or based on cellulose are also used for the preparation of spherical blank supports.

The active principle can also be made into the form of microgranules by a process known per se such as, for example, extrusion-spheronization, coating of the active principle in a perforated turbomixer, in a fluidized bed and others.

Once obtained, these microgranules are possibly coated in a turbomixer or fluidized bed.

The various processes for production of microgranules by dry or wet granulation, presented in “Remington's pharmaceutical Sciences, 16^{th} Ed, 1980, Mack Publ. Co. of Easton, Pa., USA” can be utilized in the present invention.

The active principle can be coated with a polymer selected on the basis of the type of release desired (immediate, controlled or delayed) or its taste-masking properties.

The active principle is next combined with at least one agent making it possible to combat surreptitious administration of chemicals, and with at least one pharmaceutically acceptable excipient.

Advantageously, the invention is suitable for any pharmaceutical form, in particular for an oral form, selected from non-coated tablets such as conventional tablets, suckable tablets, sublingual tablets, chewable tablets, effervescent tablets, dispersible tablets, orodispersible tablets, a powder for sachets or gel capsules, and thin films.

The invention is more especially useful for immediate release pharmaceutical compositions, since the criminal will want the effect of loss of vigilance to be as rapid as possible. It could however be adapted to controlled release forms.

Those skilled in the art know how to adapt the formulation depending on the pharmaceutical form and the desired release.

The pharmaceutically acceptable excipients utilized in the pharmaceutical compositions according to the invention are conventionally used excipients.

The following may for example be cited:
binders: for example cellulose derivatives such as HPMC, in particular the grades Pharmacoat® 603 and Pharmacoat® 606, or hydroxypropylcellulose or hydroxyethylcellulose, microcrystalline cellulose, polyvinylpyrrolidone derivatives, in particular the PVP K 30 grade, polyethylene glycol derivatives, in particular polyethylene glycol the molecular weight whereof is between 600 and 7000, such as PEG4000 and PEG6000 in particular, and mixtures thereof, and vinyl derivatives such as polyvinyl alcohol;
diluents: for example soluble diluents such as lactose or mannitol, and cellulose derivatives such as microcrystalline cellulose;
preservatives: for example parabens, and antioxidants such as ascorbic acid;
solubilizers: for example poloxamers and cyclodextrins;
disintegrants: for example crospovidone and croscarmellose sodium;
sweeteners: such as aspartame and acesulfam potassium;
lubricants: magnesium stearate, sodium stearylfumarate and cotton oil;
flavors: such as mint, lemon, black cherry flavor, etc.;
surfactants: alkali or alkaline earth metal salts of fatty acids, sodium dodecyl sulfate and sodium docusate, polyethoxylated oils, preferably polyethoxylated hydrogenated castor oil, polyoxy-ethylene-polyoxypropylene copolymers, polyethoxylated sorbitan esters, polyethoxylated castor oil derivatives, stearates, preferably of calcium, magnesium, aluminum or zinc, polysorbates, stearyl-fumarates, preferably sodium, glycerol behenate, benzalkonium chloride, acetyltrimethylammonium bromide, cetyl alcohol and mixtures thereof; and
glidants: for example silica, talc and mixtures thereof.

In the context of the present invention, orodispersible tablet is understood to mean a “multiparticulate tablet disintegrating in the mouth on contact with the saliva in less than 40 seconds”. According to a particular embodiment, the invention relates to such a tablet which is based on a mixture of excipients and particles of coated active principle exhibiting intrinsic tableting properties. The mixing proportion of excipients relative to the particles of coated active principle is from 0.4 to 6, preferably from 1 to 4 parts by weight. The mixture of excipients comprises:
a disintegration agent or disintegrant,
a soluble diluent with binding properties,
a lubricant,
a compound making it possible to combat surreptitious administration of chemicals, selected from the group comprising opacifying agents, fluorescent agents, floating particles, particles perceptible in the mouth, effervescent microgranules, and mixtures thereof,
possibly, a permeabilizing agent, sweeteners and flavors,
and possibly a coloring agent making it possible to combat surreptitious administration of chemicals.

The proportion of disintegration agent and of soluble agent relative to the mass of the tablet being from 1 to 15%, preferably from 2 to 7% by weight for the first and from 30 to 90%, preferably from 40 to 70% by weight for the second.

The soluble diluent with binding properties consists of a polyol with fewer than 13 carbon atoms taking either the form of the directly tabletable product the mean diameter of the particles whereof lies between 100 and 500 micrometers, or the form of a powder the mean diameter of the particles whereof is less than 100 micrometers, this polyol preferably being selected from the group comprising mannitol, xylitol, sorbitol and maltitol, the sorbitol not being usable alone.

The disintegration agent is selected from the group comprising in particular the crosslinked sodium carboxymethylcellulose known in the trade by the term croscarmellose, crospovidone and mixtures thereof. Through the choice and the proportion of this disintegration agent, the tablet retains an acceptable hardness for normal tablet handling conditions when they are kept in sealed packaging up to temperatures of at least 30° C.

The lubricant preferably utilized in this mixture of excipients is selected from the group comprising magnesium stearate, sodium stearylfumarate, stearic acid, micronized polyoxyethylene glycol (micronized Macrogol 6000) and mixtures thereof. It can be utilized in a proportion of 0.05 to 2% relative to the total mass of the tablet.

As permeabilizing agent, a compound selected from the group comprising in particular silicas having a high affinity for aqueous solvents, such as the precipitated silica better known under the brand name Syloid, maltodextrins, 1-cyclodextrins and mixtures thereof is used.

The permeabilizing agent enables the creation of a hydrophilic network which facilitates the penetration of the saliva and thus contributes to better disintegration of the tablet.

The various compounds and production processes for orodispersible tablets described in FR2785538, WO0027357, FR2679451, WO 93/01805, FR2766089, WO 00/51568, FR2790387, WO 03/039520 and FR2831820 can be utilized in the present invention.

The invention will be described in more detail below, in particular by means of examples which are given solely by way of illustration.

### EXAMPLES

### Example 1

Orodispersible tablets containing 10 mg of zolpidem and an opacifying agent and having the following composition are prepared:

| Constituents | % | mg/unit |
|---|---|---|
| Zolpidem grains | 32.8 | 82.1 |
| Microcrystalline cellulose | 9.6 | 23.90 |
| Mannitol | 30.0 | 75.00 |
| Crospovidone | 5.0 | 12.50 |
| Calcium silicate | 20.0 | 50.00 |
| Aspartame | 1.0 | 2.50 |
| Flavor | 0.1 | 0.25 |
| Silica | 1.0 | 2.50 |
| Mg stearate | 0.5 | 1.25 |
| Total | 100.0 | 250.0 |

The orodispersible tablets are prepared as follows.

Firstly the zolpidem grains which have the following percentage composition are prepared:

| | |
|---|---|
| NPTAB 190 (180-220 μm) | 56 |
| Zolpidem tartrate | 13 |
| Hypromellose 603 | 7 |
| 1N HCl | 2 |
| Aquacoat ECD30 | 13 |
| Hypromellose 603 | 6 |
| Triethyl citrate | 3 |

The zolipidem tartrate is dissolved in water with the aid of HCl, then a dispersion is prepared by addition of hypromellose 603. NPTAB 190 sugar spheres and the dispersion prepared above are introduced into a GPCG1 fluidized bed (Glatt). An aqueous dispersion of aquacoat ECD30, triethyl citrate and hypromellose 603 is then introduced to obtain a taste-masking coating.

The zolpidem grains are then mixed with the tableting excipients. The powdery mixture is then tableted on a rotary tablet press (SVIAC PR12) equipped with round, convex punches, at a compression force of 5 kN.

250 mg tablets of 8.5 mm diameter are obtained which have the following properties:

Hardness: 38 N

Disintegration (measured according to monograph 2.9.1 of European Pharmacopeia 6.1): 15 secs

Friability (measured according to monograph 2.9.7 of European Pharmacopeia 6.1): 0.32%

The tablets exhibit a pleasant mouth feel.

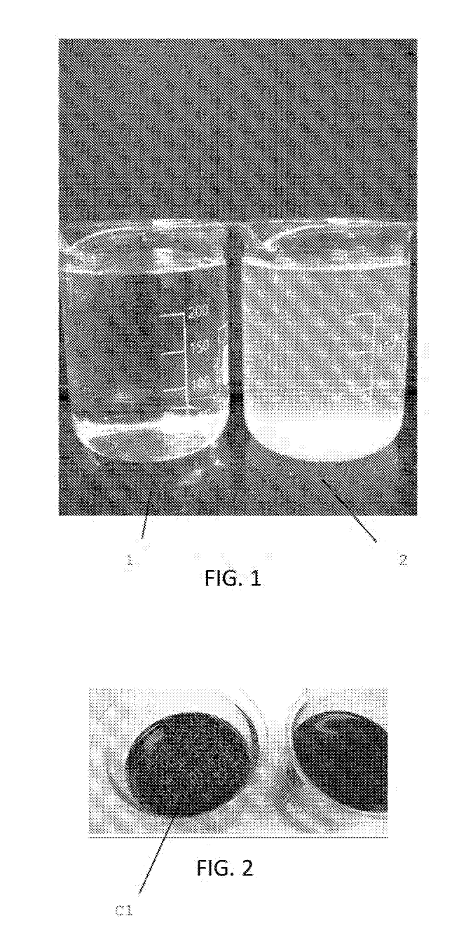

One tablet is introduced into a transparent vessel containing 250 ml of water. A cloudiness appears as soon as the tablet is disintegrated, as illustrated in FIG. 1.

FIG. 1 is a photograph showing a beaker containing 250 ml of water (1) and a beaker into which the tablet prepared in this example has been introduced (2).

### Example 2

Conventional zolpidem tablets containing an opacifying agent and having the following general formula are prepared:

| | % | Mg/unit |
|---|---|---|
| Zolpidem grains | 32.8 | 82.0 |
| Microcrystalline cellulose | 10.0 | 25.0 |
| Lactose | 32.7 | 81.75 |
| Calcium silicate | 20.0 | 50.0 |
| Povidone | 3.0 | 7.5 |
| Silica | 1.0 | 2.50 |
| Mg stearate | 0.5 | 1.25 |
| Total | 100.0 | 250.0 |

The zolpidem grains are prepared in the same way as in example 1 above. They are then mixed with the excipients and the powdery mixture is then tableted.

One tablet thus prepared is introduced into a glass of water in which it dissolves forming a cloudiness very visible to the naked eye.

### Example 3

Two types of orodispersible tablet containing floating particles and having the following formula are prepared:

| Constituents | % | mg/unit |
|---|---|---|
| Zolpidem grains | 32.8 | 82.1 |
| Microcrystalline cellulose | 9.6 | 23.90 |
| Mannitol | 30.0 | 75.00 |
| Crospovidone | 5.0 | 12.50 |
| Floating particles | 20.0 | 50.00 |
| Aspartame | 1.0 | 2.50 |
| Flavor | 0.1 | 0.25 |
| Silica | 1.0 | 2.50 |
| Mg stearate | 0.5 | 1.25 |
| Total | 100.0 | 250.0 |

These tablets are prepared as in example 1, except that the calcium silicate is replaced by floating particles.

For the first series of tablets (C1), the floating particles are prepared as follows:

NPTAB 190 (180-220 μm) blanks are coated with an aqueous dispersion of ethylcellulose, triacetin and talc. The coating factor is 30% of dry mass and the talc/polymer ratio is 1:2.

For the second series of tablets (C2), the floating particles are particles of dibasic calcium phosphate dihydrate coated with glyceryl palmitostearate. The glyceryl palmitostearate/dibasic calcium phosphate dihydrate ratio is 1:4.

The tablets of both series disintegrate in less than 30 secs, and exhibit a pleasant mouth feel.

One tablet of each type is introduced into a glass of coca-cola. The disintegration takes place immediately and the presence of particles on the surface of the coca-cola is detectable with the naked eye. These floating particles are visible on the surface for more than 3 hours.

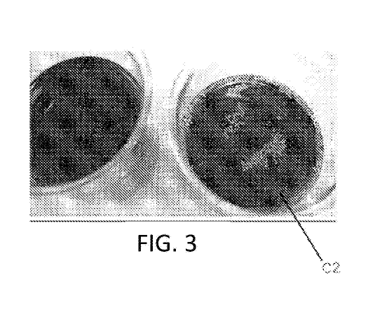

The results are presented in FIGS. 2 and 3 which show respectively a photograph of a glass of coca-cola and a glass of coca-cola into which one tablet C1 or one tablet C2 has been introduced. The photographs were taken 5 minutes after the introduction of the tablet. The presence of the floating particles was nonetheless detectable much more rapidly, about 30 seconds after the introduction of the tablets.

### Example 4

Immediate release morphine gel capsules containing floating particles are prepared as follows:

SP sugar blanks (400-600 μm) are introduced into a GPCG1 fluidized bed (Glatt) and an aqueous dispersion of morphine sulfate and hypromellose 603 is sprayed onto them so as to obtain the following percentage composition: 43% of SP blank, 42% of morphine sulfate and 15% of hypromellose.

Separately, floating particles of dibasic calcium phosphate dihydrate coated with glyceryl palmito-stearate are prepared as mentioned in example 3.

40 mg of floating particles and the quantity of morphine grains necessary to obtain the desired dosage 10 mg, 20 mg or 30 mg of morphine are introduced into gel capsules.

The two populations of particles present in the gel capsule are not distinguishable to the naked eye.

When the contents of one of these gel capsules are introduced into a drink, the floating particles become immediately apparent on the surface of the drink.

### Example 5

Morphine sulfate gel capsules are prepared as in the previous example but using blanks of 250-300 μm diameter and replacing the floating particles with floating particles prepared as follows: NPTAB blanks (300-350 μm) are coated with an aqueous dispersion of ethylcellulose, triacetin and talc. The coating factor is 40% of dry mass and the talc/polymer ratio is 1:2.

The two populations of particles are introduced into the gel capsules. The two populations of particles are not distinguishable to the naked eye.

The contents of one gel capsule are introduced into a bottle of coca-cola. The floating particles immediately rise to the surface and as soon as the person brings the bottle to their mouth they feel the presence of the microparticles, a sign that a substance has been introduced into the bottle.

### Example 6

Orodispersible tablets containing 5 mg of zolpidem and a fluorescent agent and having the following composition are prepared:

| | % | Mg/unit |
|---|---|---|
| Zolpidem grains | 32.8 | 41.05 |
| Microcrystalline cellulose | 10.0 | 12.50 |
| Mannitol | 43.7 | 54.57 |
| Crospovidone | 10.0 | 12.50 |
| Aspartame | 1.0 | 1.25 |
| Flavor | 0.1 | 0.13 |
| Fluorescein | 0.4 | 0.50 |
| Silica | 1.0 | 1.25 |
| Mg stearate | 1.0 | 1.25 |
| Total | 100.0 | 125.0 |

The orodispersible tablets are prepared as follows.

Firstly, the zolpidem grains which have the following composition are prepared:

| | |
|---|---|
| NPTAB 190 (180-220 μm) | 56 |
| Zolpidem tartrate | 13 |
| Hypromellose 603 | 7 |
| 1N HCl | 2 |
| Aquacoat ECD30 | 13 |
| Hypromellose 603 | 6 |
| Triethyl citrate | 3 |

The zolpidem tartrate is dissolved in water with the aid of HCl, then a dispersion is prepared by addition of hypromellose 603. NPTAB 190 sugar spheres and the dispersion prepared above are introduced into a GPCG1 fluidized bed (Glatt). An aqueous dispersion of aquacoat ECD30, triethyl citrate and hypromellose 603 is then introduced to obtain a taste-masking coating.

The zolpidem grains are then mixed with the tableting excipients. The powdery mixture is then tableted on a rotary tablet press (SVIAC PR12) equipped with round, convex punches, at a compression force of 5 kN.

125 mg tablets of 7 mm diameter are obtained which have the following properties:

Hardness: 24 N

Disintegration (measured according to monograph 2.9.1 of European Pharmacopeia 6.1): 15 secs

Friability (measured according to monograph 2.9.7 of European Pharmacopeia 6.1): 0.03%

The tablets exhibit a pleasant mouth feel.

One tablet is introduced into a transparent vessel containing 250 ml of water. The surface of the water becomes fluorescent as soon as the tablet is disintegrated.

### Example 7

Orodispersible tablets containing 10 mg of zolpidem, floating particles and a fluorescent agent and having the following composition are prepared:

| | mg/unit | % |
|---|---|---|
| Zolpidem tartrate grains* | 80.0 | 26.7 |
| Avicel PH 200 | 30.0 | 10.0 |
| Mannitol SD 200 | 99.7 | 33.2 |
| Floating particles | 50.0 | 16.7 |
| Kollidon CL | 30.0 | 10.0 |
| Aspartame | 3.0 | 1.0 |
| Black cherry flavor | 0.3 | 0.1 |
| Sodium fluorescein | 1.0 | 0.33 |
| Syloid 244 FP | 3.0 | 1.0 |
| Mg stearate | 3.0 | 1.0 |
| Total | 300.0 | 100.0 |

These tablets are prepared as in example 1, with the exception that the calcium silicate is replaced by floating particles and sodium fluorescein.

The floating particles are prepared as follows: NPTAB 190 (180-220 μm) blanks are coated with an aqueous dispersion of ethylcellulose and Myvacet® (acetylated monoglyceride) 9-45. The coating factor is 30% of dry mass and the plasticizer/polymer ratio is 24%.

The tablets disintegrate in less than 15 secs, and exhibit a pleasant mouth feel.

One tablet is introduced into a glass of coca-cola. The disintegration takes place immediately and the presence of particles on the surface of the coca-cola is detectable with the naked eye. These floating particles are visible on the surface for more than 3 hours.

1. A method of enabling the immediate detection of a pharmaceutical form illicitly introduced into a drink, said pharmaceutical form comprising an active principle which modifies the state of consciousness of a person, said method comprising:
introduction into a drink of a pharmaceutical form comprising:
an active principle which modifies the state of consciousness of a person,
particles having a total diameter greater than 500 μm and a density less than one and that float in the drink and are perceptible in the mouth, said particles being microgranules comprising an insoluble blank support or a blank support coated with an insoluble polymer selected from the group consisting of non-water-soluble cellulose derivatives, (meth)acrylic (co)-polymer derivatives, polyvinyl acetate and mixtures thereof, or coated with a lipid material selected from the group consisting of glyceryl palmitostearate, waxes, polyoxyl-glycerides and glyceryl behenate,
a disintegration agent or disintegrant selected from the group consisting of crosslinked sodium carboxymethylcellulose known in the trade by the term croscarmellose, crospovidone and mixtures thereof,
a soluble diluent with binding properties selected from the group consisting of mannitol, xylitol, sorbitol, maltitol and mixtures thereof, provided that sorbitol is not the sole soluble diluent, and
a lubricant selected from the group consisting of magnesium stearate, sodium stearylfumarate, stearic acid, micronized polyoxyethylene glycol (micronized macrogol 6000) and mixtures thereof,
said pharmaceutical form being an orodispersible tablet,
wherein modification of the organoleptic properties of the drink takes place in less than one minute from the introduction of said pharmaceutical form into said drink.

2. The method according to claim 1, wherein the modification of the organoleptic properties of the drink takes place in less than 30 seconds.

3. The method according to claim 1, wherein the modification of the organoleptic properties of the drink takes place in less than 15 seconds.

4. The method according to claim 1, wherein:
the non-water-soluble cellulose derivative is selected from the group consisting of ethylcellulose, cellulose acetate butyrate, cellulose acetate,
the (meth)acrylic (co)-polymer derivative is selected from the type A and type B ammoniomethacrylate copolymers sold under the trade name Eudragit®.

5. The method according to claim 1, wherein the dry mass of the coating layer is between 0.1% and 50% of the total dry mass of the microgranule before coating.

6. The method according to claim 5, wherein the dry mass of the coating layer is between 5% to 40% of the total dry mass of the microgranule before coating.

7. The method according to claim 1, wherein the particles perceptible in the mouth are present in a quantity of at least 25 mg.

8. The method according to claim 1, wherein the particles perceptible in the mouth have a total diameter greater than 1 mm.

9. The method according to claim 1, wherein the active principle is selected from the group consisting of anxiolytics, hypnotics, sedatives and analgesics.

10. The method according to claim 1, wherein the pharmaceutical form further contains an opacifying agent which is an inorganic compound and/or a fluorescent agent.

11. The method according to claim 10, wherein the inorganic compound is present and is selected from the group consisting of silicates, titanium dioxide and mixtures thereof.

12. The method according to claim 11, wherein the silicates are present and are selected from the group consisting of magnesium silicate, aluminium silicate, magnesium aluminium silicate and calcium silicate.

13. The method according to claim 10, wherein the opacifying agent is present in a quantity from 15 mg to 100 mg.

14. The method according to claim 13, wherein the opacifying agent is present in a quantity from 20 to 60 mg.

15. The method according to claim 10, wherein the fluorescent agent is present and is selected from the group consisting of fluorescein and derivatives thereof and indocyanine green.

16. The method according to claim 10, wherein the fluorescent agent is present in a quantity of at least 0.1 mg.

17. The method according to claim 16, wherein the fluorescent agent is present in a quantity from 0.2 to 5 mg.
